(19) United States
(12) Patent Application Publication
(10) Pub. No.: US 2019/0083674 A1
(43) Pub. Date: Mar. 21, 2019

(54) MULTIFUNCTIONAL PHOTOCATALYTIC MODULE

(71) Applicant: SEOUL VIOSYS CO., LTD., Gyeonggi-o (KR)

(72) Inventors: Jae Hak Jeong, Ansan-si (KR); Hyun Su Song, Ansan-si (KR); Jong Hyun Koo, Ansan-si (KR)

(21) Appl. No.: 15/533,334

(22) PCT Filed: Dec. 1, 2015
(86) PCT No.: PCT/KR2015/013004

§ 371 Date: Jun. 5, 2017

(30) Foreign Application Priority Data
Dec. 4, 2014 (KR) 10-2014-0173019

(51) Int. Cl. A61L 9/20; B01D 53/88; B01D 53/00; F25D 17/04

(52) U.S. Cl. CPC A61L 9/205; B01D 53/885; B01D 53/007; F25D 2317/0417; B01D 2255/802; A61L 2209/14; A61L 2209/12; F25D 17/042

(57) ABSTRACT

The disclosed invention relates to a multifunctional photocatalytic module which includes a duct 10 having a flow cross section with long sides 11 and short sides 12; a suction port 13 and a discharge port 14, the suction port 13 and the discharge port 14 being informed on both ends of the duct; a fan 20 disposed close to the suction port in the duct, the fan introducing air from the suction port and apply pressure to the air toward the discharge port; a photocatalytic filter 40 disposed close to the discharge port in the duct; and a light source disposed between the photocatalytic filter 40 and the fan 20 and configured to radiate ultraviolet light toward the photocatalytic filter.

### TECHNICAL FIELD

The disclosed invention relates to a multifunctional photocatalytic module and, more particularly, to a multifunctional photocatalytic module which is installed in a space such as the interior of the refrigerator containing food and serves to deodorize and sterilize the interior of the refrigerator, to maintain freshness of fruits or vegetables and to decompose hazardous gases emitted into the air in a new refrigerator.

### BACKGROUND ART

When air present in a food storage space such as a refrigerator is mixed with odors of food, it causes great uncomfortableness to a user. Currently, deodorizers equipped with activated carbon for adsorbing unpleasant odors are commercially available in the market. Consumers having purchased deodorizers leave the deodorizers in the refrigerator for a certain time, and then discard the same. Since such deodorizers are disposable and are difficult to reuse, inconvenience is caused by frequent replacement of the deodorizers

In contrast, a photocatalytic filter, which performs deodorization by decomposing hazardous gases in the air using a photocatalytic material activated by light such as ultraviolet light, can be reused and thus may be semi-permanently used once installed.

However, in this structure, a light source providing activation energy and a photocatalytic filter activated by ultraviolet light should be installed together, and a certain distance needs to be maintained between the photocatalytic filter and the light source. It is not easy to install these constituents in a narrow inner space of the refrigerator.

Additionally, compared to general indoor air, air in the inner space of the refrigerator is seriously contaminated by gases evaporating from food. Accordingly, the photocatalytic filter and the light source need to have enhanced efficiency with a form as compact as possible. Accordingly, a wholly new design needs to be implemented in consideration of air flow paths, an air suction direction and discharge direction, an installation structure for the filter, a relationship between the direction of installation of the filter and the air flow direction, an installation position of the light source and the like.

In addition, in arranging an ultraviolet light source and a photocatalytic filter in a narrow space, the properties of the ultraviolet light source and the photocatalytic filter need to be tuned so as to produce a sufficient effect.

Meanwhile, odors of food are not the only problem in the refrigerator. When fruits or vegetables remain stored in the refrigerator, ethylene emitted from fruits or vegetables causes the fruits or vegetables to be softened and ripened too early. But it is not reasonable to frequently open the door of the refrigerator to remove ethylene from the inner space of the refrigerator since the cooled air would leak out therefrom. Further, ethylene is not easily adsorbed by a deodorant.

In addition, microbes or germs floating in the air are densely populated in the closed inner space of the refrigerator where food is stored, but the deodorizer can not destroy microbes or germs. If the food stored in the refrigerator goes bad or spoils due to the germs or microbes, the food becomes inedible, and when eaten, it may cause stomachache or food poisoning.

Additionally, when a new refrigerator is purchased, hazardous gases such as petroleum-based aromatic compounds emitted from the synthetic resin of the inner wall may remain in the refrigerator for several years as a closed space is formed in the refrigerator.

The problem with the air in the refrigerator cannot be solved by a conventional air freshener or deodorizer.

### DISCLOSURE

### Technical Problem

It is an aspect of the disclosed invention to provide a compact multifunctional photocatalytic module capable of deodorizing the inner space of a refrigerator, maintaining freshness of vegetables or fruits, performing sterilization and removing hazardous gases using a photocatalytic filter and a light source.

It is another aspect of the disclosed invention to provide a multifunctional photocatalytic module provided with enhanced photocatalytic reaction efficiency by maximizing air flow.

It is another aspect of the disclosed invention to provide an air flow direction, an installation direction of a photocatalytic filter, a form of the photocatalytic filter, a relationship between the photocatalytic filter and a light source, and properties of the light source which may enhance photocatalytic reaction efficiency.

### Technical Solution

In accordance with one aspect of the disclosed invention, provided herein is a multifunctional photocatalytic module for inducing active movement of air by generating turbulence in a duct configured to be slim and implementing proper arrangement of a photocatalytic filter and the light source to enhance photocatalytic reaction.

The multifunctional photocatalytic module includes a duct 10 having a flow cross section with long sides 11 and short sides 12; a suction port 13 and a discharge port 14, the suction port 13 and the discharge port 14 being informed on both ends of the duct; a fan 20 disposed close to the suction port in the duct, the fan introducing air from the suction port and apply pressure to the air toward the discharge port; a photocatalytic filter 40 disposed close to the discharge port in the duct; and a light source disposed between the photocatalytic filter 40 and the fan 20 and configured to radiate ultraviolet light toward the photocatalytic filter, wherein the fan 20 discharge the air by applying pressure to the air in a direction inclined and a predetermined angle a with respect to a longitudinal direction of the duct.

The predetermined angle may be within a range between 30° and 60°.

The direction of application of pressure to the air may be directed to a surface of a first long side of the duct.

The fan may have a flat shape and be installed by being inclined at a predetermined angle a with respect to a vertical cross section of the duct in the longitudinal direction of the duct.

The light source may be installed downstream of the fan and include at least one ultraviolet (UV) light emitting diode (LED) 31 provided on a substrate 30.

A peak wavelength of the UV LED 31 may be between 360 nm and 370 nm.

A distance d1 between the first long side and an end of the substrate may be shorter than a distance d2 between a second long side facing in an opposite direction of the first long side and an end of the substrate.

A distance d3 between a surface of the photocatalytic filter facing the light source and the light source may be between 25 mm and 40 mm.

An intensity of ultraviolet light radiated onto a surface of the photocatalytic filter facing the light source may be between 12 mW/cm² and 18 mW/cm².

Inner surfaces of the duct corresponding to inner surfaces of the short sides 12 between the light source and the photocatalytic filter may be provided with a flow guide surface 15 protruding inward, wherein the long sides of a flow cross section of the duct after the flow guide surfaces may be shortened compared to the long sides of a cross section of the duct before the flow guide surfaces and have the same length as the short sides thereof.

The photocatalytic filter may be installed on a flow cross section of the duct after the flow guide surfaces.

The photocatalytic filter may be formed by applying a photocatalytic material onto a supporter having a plurality of cells neighboring each other and provided with air flow paths, inlets of the air flow paths being disposed to face the light source.

The suction port 13 may be formed at a position close to the first long side.

A nozzle part may be formed near the discharge port 14 of the duct, a flow cross section of the nozzle part being narrowed as the cross section is shifted toward the discharge port.

In accordance with another aspect of the disclosed invention, provided herein is a refrigerator provided with the multifunctional photocatalytic module, wherein the suction port and discharge port of the duct are installed to communicate with a cooling space of the refrigerator.

### Advantageous Effects

According to embodiments of the disclosed invention, a photocatalytic filter and the light source may be installed in a narrow space of the refrigerator and semi-permanently deodorize the refrigerator. In addition, air movement may be activated by creating turbulence in the air flow. Thereby, high photocatalytic reaction efficiency may be obtained despite the compact design of the module.

According to embodiments of the disclosed invention, high photocatalytic reaction efficiency may be obtained from the structure and properties of the photocatalytic filter and the light source despite the compact design of the photocatalytic module.

According to embodiments of the disclosed invention, not only deodorization is implemented, freshness of fresh fruit may be maintained for a long time.

According to embodiments of the disclosed invention, germs and microbes may be removed from the air to prevent food from spoiling.

According to embodiments of the disclosed invention, hazardous gases coming out of the material of the inner wall of the refrigerator may be removed.

The effects that can be obtained from the disclosed invention are not limited to the aforementioned effects, and other effects may be clearly understood by those skilled in the art from the descriptions given below.

### DESCRIPTION OF DRAWINGS

FIG. 1 is a perspective view illustrating a multifunctional photocatalytic module according to an embodiment of the disclosed invention.

FIG. 2 is a perspective view illustrating the multifunctional photocatalytic module of FIG. 1 with an upper cover of the duct removed.

FIG. 3 is a lateral cross-sectional view of FIG. 1.

FIG. 4 is a plan view illustrating the multifunctional photocatalytic module of FIG. 2.

FIG. 5 is a graph depicting results of an experiment of trimethylamine removal performance of a multifunctional photocatalytic module according to an embodiment of the disclosed invention.

FIG. 6 is a graph depicting a result of an experiment of ethylene removal performance of a multifunctional photocatalytic module according to an embodiment of the disclosed invention.

FIG. 7 is a graph depicting results of an experiment of freshness maintenance performance of a multifunctional photocatalytic module conducted for broccoli according to an embodiment of the disclosed invention.

FIG. 8 is a graph depicting results of an experiment of freshness maintenance performance of a multifunctional photocatalytic module conducted for kale according to an embodiment of the disclosed invention.

FIG. 9 is a graph depicting results of an experiment of air sterilization performance of a multifunctional photocatalytic module according to an embodiment of the disclosed invention.

### MODE FOR INVENTION

Hereinafter, exemplary embodiments of the disclosed invention will be described in detail with reference to the accompanying drawings.

The disclosed invention is not limited to exemplary embodiments disclosed herein but may be implemented in various different forms. The exemplary embodiments are provided for making the disclosure of the disclosed invention thorough and for fully conveying the scope of the disclosed invention to those skilled in the art.

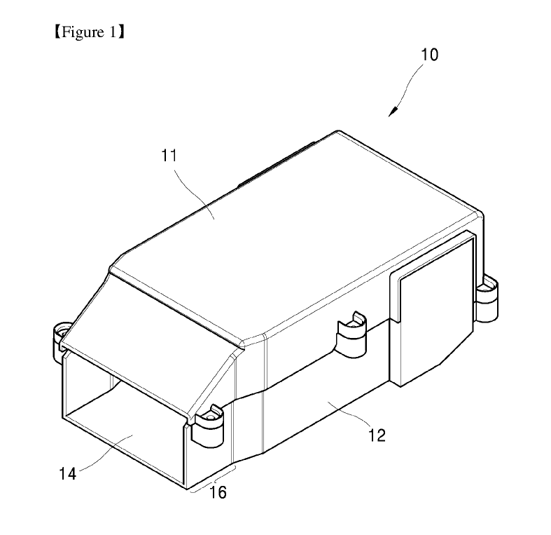

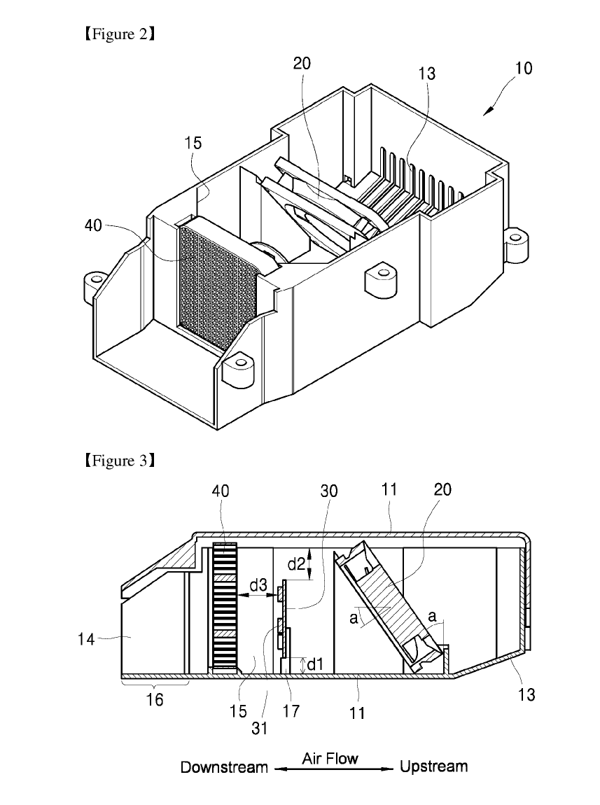

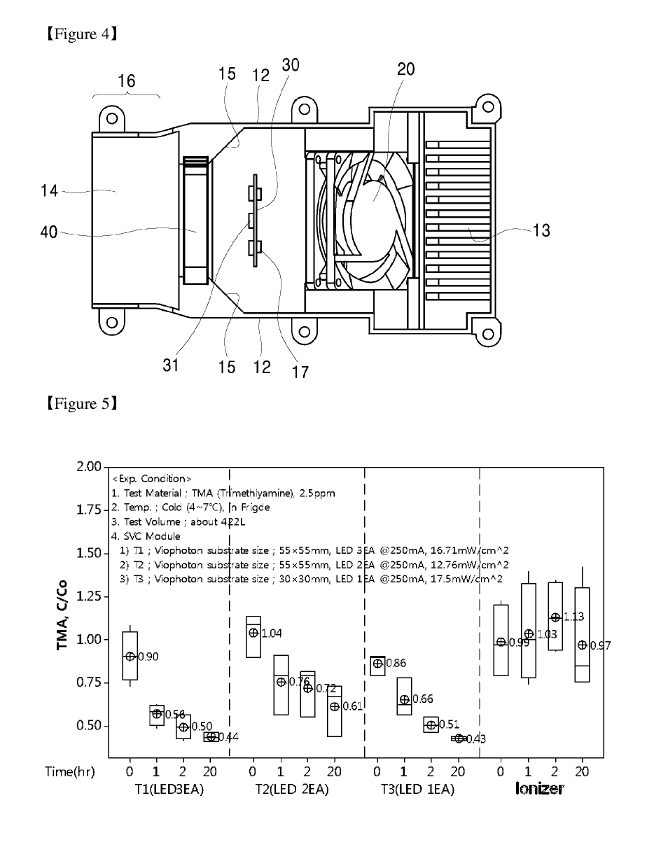

FIG. 1 is a perspective view illustrating a multifunctional photocatalytic module according to an embodiment of the disclosed invention. FIG. 2 is a perspective view illustrating the multifunctional photocatalytic module of FIG. 1 with the upper cover of the duct removed. FIG. 3 is a lateral cross-sectional view of FIG. 1. FIG. 4 is a plan view illustrating the multifunctional photocatalytic module of FIG. 2.

The multifunctional photocatalytic module of the disclosed invention is configured to be slim and compact as a component installed in a refrigerator. A duct 10 has a flow cross section formed by long sides 11 and short sides 12. The duct 10 generally has a flat and long shape.

Both ends of the duct 10 are provided with a suction port 13 and a discharge port 14. Air in the refrigerator is introduced into the duct through the suction port, purified therein and then discharged through the discharge port.

Installed in the duct are a fan 20, a photocatalytic filter 40 and a substrate 30 provided with an ultraviolet (UV) light emitting diode (LED) 31 as a light source. The fan 20 is positioned in the uppermost stream in the duct. That is, the fan 20 is disposed close to the suction port to suction air through the suction port and apply pressure to the air to discharge the air toward the discharge port. The photocatalytic filter 40 is disposed close to the discharge port 14, and is formed by applying or coating TiO₂, which is a photocatalytic material, onto a supporter formed by a plurality of cells adjacent to each other and provided with air flow paths. The photocatalytic filter 40 is installed such that the air flow paths are aligned with a path along which air flows. Thereby, the air flowing in the duct is discharged outside, passing through the air flow paths defined by the respective cells of the photocatalytic filter.

The substrate 30 is disposed between the fan 20 and the photocatalytic filter 40 and arranged in the duct in a direction in which the UV LED 31 formed on the substrate emits light toward the photocatalytic filter 40. That is, the substrate 30 is disposed upstream from the photocatalytic filter 40.

As the fan 20, a flat square-shaped fan may be used. The square-shaped fan is allowed to be installed in the duct having a rectangular flow cross section when the fan is slightly inclined. Accordingly, the fan installed in the duct is inclined at a predetermined angle with respect to the vertical cross section of the duct in the longitudinal direction of the duct. If the fan is arranged inclined in the lateral direction of the duct, a large fan cannot be installed in the duct. In this case, pressure applied to the air is lowered, and air flow cannot be properly distributed through the gap between the substrate 30, which will be described later, and the inner wall of the duct. Further, proper air flow characteristics cannot be provided to a flow guide surface 15 defining the square-shaped flow cross section.

On the other hand, a structure causing the fan to be inclined at a predetermined angle a in the longitudinal direction allows a square-shaped fan having a good air pressurization efficiency to be installed in a slim duct. Moreover, this structure applies pressure to the air in a direction inclined at the predetermined angle a with respect to the longitudinal direction of the duct to generate air flow. Accordingly, turbulence causing irregular and active air flow may be generated.

Preferably, the predetermined angle a is determined in a range between 30° and 60°. If the inclination angle is less than 30°, efficiency of turbulence generation is lowered and the duct cannot be formed to be slim. If the angle is greater than 60°, the air flow direction and the longitudinal direction of the duct become excessively misaligned and thus air flow efficiency decreases drastically.

The substrate 30 is installed downstream of the fan at a position spaced from all inner surfaces of the duct 10. The substrate 30 is fixed to an installation portion 17 protruding from an inner surface of the duct. A distance d2 between a first long side facing the fan and an end of the substrate is set to be shorter than a distance d2 between a second long side and the other end of the substrate such that the amount of air passing a space near the first long side, on which air flow is concentrated, is balanced with the amount of air passing a space near the second long side, on which air flow is not concentrated. This configuration is advantageous in enhancing deodorization efficiency of the photocatalytic filter. Meanwhile, the distances from the substrate 30 to short sides are equal to each other as shown in FIG. 4.

As shown in the figure, at least one UV LED 31, whose dispersion angle is approximately around 120°, is installed on the substrate 30 and radiates ultraviolet light toward the photocatalytic filter 40. The photocatalytic filter 40 is formed by fixing a photocatalytic material to a supporter. In this embodiment, titanium dioxide is used as the photocatalytic material.

The ultraviolet light absorption rate of the TiO₂ photocatalytic filter according to ultraviolet light wavelengths has a peak value when the ultraviolet light wavelength is around 270 nm, and then linearly decreases as the wavelength increases to 400 nm. Accordingly, it may appear that using a UV LED having 270 nm as the peak wavelength is favorable. However, when the LEDs are used in reality, it is found that best photocatalytic activation is obtained with a UV LED having 365 nm as the peak wavelength. This result is related to light emission efficiency of UV LEDs. That is, as the peak wavelength decreases, the amount of light emitted from the UV LED decreases drastically. Accordingly, the best photocatalytic reaction can be obtained when a UV LED having a peak wavelength equal to 365 nm is used in reality.

In other words, for a UV LED having a peak wavelength which is around 270 nm, intensity of emitted ultraviolet light is very low compared to a proper intensity of ultraviolet light required on the surface of the photocatalytic filter, and thus photocatalytic reaction is not active. If the number of UV LEDs is increased to increase the intensity of ultraviolet light in consideration of this fact, the size of the substrate may increase and interrupt the air flow. Further, increasing the number of UV LEDs is not preferable since it drastically increases manufacturing costs and power consumption. Furthermore it would generate heat quite a lot, which is not desirable as a refrigerator component.

It was found in an experiment that deodorization efficiency of the photocatalytic filter was remarkably lowered when a UV LED having a peak wavelength less than or equal to 340 nm was used.

In addition, if a UV LED having a peak wavelength greater than or equal to 380 nm is used, the ultraviolet light absorption rate of photocatalytic reaction is significantly lowered and it comes to have no difference from that of a conventional ultraviolet lamp such as black light and thus use of the UV LED becomes meaningless.

The results of the experiment show that the highest deodorization performance of the photocatalytic filter can be obtained when a UV LED having a peak wavelength between 360 nm and 370 nm is used.

The photocatalytic filter has a structure in which a photocatalytic material is applied onto a supporter constructed by a plurality of cells forming an airflow path having a hexagon-shaped or square-shaped cross section similar to a honeycomb shape. The inlet of the air flow path is disposed in the flow direction of air to face an ultraviolet light source as shown in FIGS. 2 and 3. As the photocatalytic filter is fabricated in this form, ultraviolet light can be emitted not only onto the outer surface of the photocatalytic filter but also onto the inner surface of the air flow path. Thereby, photocatalytic reaction may be boosted.

The distance between the UV LED 31 and the front face of the photocatalytic filter 40 facing the same depends on change in flow characteristics of air according to the distance between the UV LED substrate and the photocatalytic filter and the area and intensity of ultraviolet light reaching the photocatalytic material. It can be seen from the results of the experiment that deodorization efficiency decreases significantly when the distance between the UV LED and the front face of the photocatalytic filter decreases below 2.5 cm or increases beyond 4 cm.

If the distance between the UV LED and the front face of the photocatalytic filter decreases below 2.5 cm, the area of the photocatalytic filter onto which ultraviolet light is emitted decreases, and intensity of ultraviolet light per unit area of the photocatalytic filter increases, while photocatalytic activation efficiency does not increase anymore. Additionally, if the UV LED substrate is disposed excessively close to the photocatalytic filter, air rarely flows through the middle region of the photocatalytic filter, onto which ultraviolet light is mainly radiated. Accordingly, as the amount of air contacting the region where photocatalytic activation occurs most actively decreases, deodorization efficiency of the photocatalytic filter is degraded. If the distance between the UV LED and the front face of the photocatalytic filter increases beyond 4 cm, intensity of ultraviolet light per unit area of the photocatalytic filter decreases, and thus the degree of photocatalytic activation is lowered.

It is necessary to take into consideration the intensity of ultraviolet light reaching the photocatalytic filter. It may be thought that high intensity of ultraviolet light reaching the surface of the photocatalytic filter will increase the photocatalytic reaction efficiency. However, the experiment shows that the photocatalytic reaction efficiency increases along with intensity of ultraviolet light only to a certain level, and then does not increase anymore even if the intensity is further increased. It can be seen from the result of the experiment that increasing the ultraviolet light intensity beyond about 18 mW/cm² significantly slows trend of increase of the photocatalytic reaction efficiency for a UV LED having a peak wavelength between 360 nm and 370 nm. In addition, if the intensity of ultraviolet light is less than about 12 mW/cm², the intensity of ultraviolet light is insufficient and thus the photocatalytic reaction efficiency is significantly lowered.

The flow direction of air also needs to be considered. In this embodiment, the flow direction of air is identical to the direction in which the UV LED serving as the ultraviolet light source faces the photocatalytic filter.

This arrangement is based on the result of an experiment. It has been found from the experiment that driving air to flow in the same direction as the direction in which the ultraviolet light source faces the photocatalytic filter obtains even higher purification efficiency than driving the air to flow in the opposite direction.

Since the photocatalytic filter has a structure with multiple air flow paths through which air is guided, air pressure is lowered due to flow resistance generated as the air passes through the photocatalytic filter. Meanwhile, photocatalytic reaction is promoted when the surface of the photocatalytic material contacts as much air as possible. Accordingly, higher decomposition efficiency for a hazardous gas in the air can be obtained when air contacts the photocatalytic material before pressure drop occurs in the air through the photocatalytic filter than when the air contacts the photocatalytic material after air pressure drops through the photocatalytic filter. Accordingly, in this embodiment, the air is caused to flow in the direction in which the ultraviolet light source faces the photocatalytic filter, in order to enhance the air purification efficiency of the photocatalytic filter.

In this embodiment, turbulence is generated with reduced flow loss of air flowing in the duct, as described above. Accordingly, efficiency of contact between the photocatalytic filter and the air increases, thereby enhancing the air purification efficiency.

In this embodiment, the photocatalytic filter 40 having a square shape is used. The flow cross section of air formed in the duct has the shape of a rectangle having long sides and short sides. Accordingly, using a photocatalytic filter 40 having a corresponding rectangular shape may be considered. However, if the flow guide surface 15 protruding inward from inner surfaces of the duct on the short sides 12 between the light source and the photocatalytic filter is provided such that the flow cross section of the duct becomes close to a square shape as it moves through the flow guide surface, and then a square-shaped photocatalytic filter 80 is installed in the flow cross section, UV LEDs 31 for radiating light more uniformly onto the square shape than onto the rectangular shape may be disposed more closely. Thereby, substrate may become more compact, and thus flow resistance by the substrate may be significantly reduced. Further, air flow accelerated by the flow guide surface may remarkably increase the efficiency of contact between the photocatalytic filter and the air, thereby further enhancing purification efficiency compared to a rectangular photocatalytic filter.

The multifunctional photocatalytic module of the disclosed invention generates turbulence while minimizing flow loss in a slim duct. To enhance efficiency of generation of turbulence, the suction port 13 is formed at a position close to the first long side. This structure causes air introduced from the first long side into the duct to be pressurized against the first long side by the fan, thereby generating tumbling currents. Accordingly, turbulent currents are prevented from colliding with each other to consume kinetic energy.

In addition, the multifunctional photocatalytic module of the disclosed invention needs to purify air in the refrigerator in which various obstacles containing food (in view of air flow) are accommodated. Accordingly, it is better to smoothly circulate the air in the refrigerator and introduce the same into the multifunctional photocatalytic module rather than letting the air remain calm. Therefore, air is preferably discharged from the discharge port 14 at a considerable flow velocity.

To this end, a nozzle part 16 whose flow cross section is narrowed as the position is shifted toward the discharge port is formed near the discharge port 14 of the duct. As shown in the figure, the nozzle part may be configured such that the long side portion of the duct is inclined. The shape of the nozzle part is not limited to the illustrated shape.

The multifunctional photocatalytic module of the disclosed invention is installed such that the suction port and the discharge port communicate with the cooling space of the refrigerator. Thereby, the multifunctional photocatalytic module purifies the air in the cooling space. However, if external warm air enters the interior of the refrigerator as the door of the refrigerator is opened and closed, moisture is condensed on the inner wall of the refrigerator. This phenomenon may also occur in the inner space of the duct.

The moisture may adversely affect the electrical structure including the UV LED. Accordingly, when the multifunctional photocatalytic module of the disclosed invention is installed in the refrigerator, a heat source for maintaining the temperature of the interior of the duct to be higher than the temperature of the cooling space of the refrigerator is preferably installed inside or outside the duct.

The heat source may employ, for example, the heat source of a sealing portion (not shown) for sealing the space between the refrigerator door and the refrigerator body.

### EXPERIMENTAL EXAMPLE

FIG. 5 is a graph depicting results of an experiment of trimethylamine removal performance of a multifunctional photocatalytic module according to an embodiment of the disclosed invention.

Trimethylamine is one of odor molecules produced from food. In this embodiment, the volume of the closed space is 422 L, a photocatalytic filter having a surface area of 55² mm² or 30² mm² and power of 250 mA is applied for the air mixed with trimethylamine of 2.5 ppm at a cooling temperature between 4° C and 7° C. Under these conditions, the experiment was conducted by changing the number of UV LEDs having a peak wavelength of 365 nm such that the intensity of ultraviolet light measured on the surface of the filter ranges from 12 mW/cm²to 18 mW/cm². All the results of the experiment differ from each other to a certain degree, but showed high trimethylamine removal performance. A commercialized ionizer, on the other hand, did not have a deodorization effect. That is, with the multifunctional photocatalytic module of the disclosed invention, high freshness maintenance efficiency for fresh food is obtained.

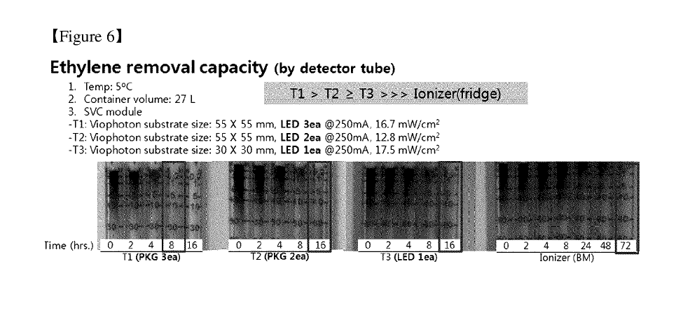

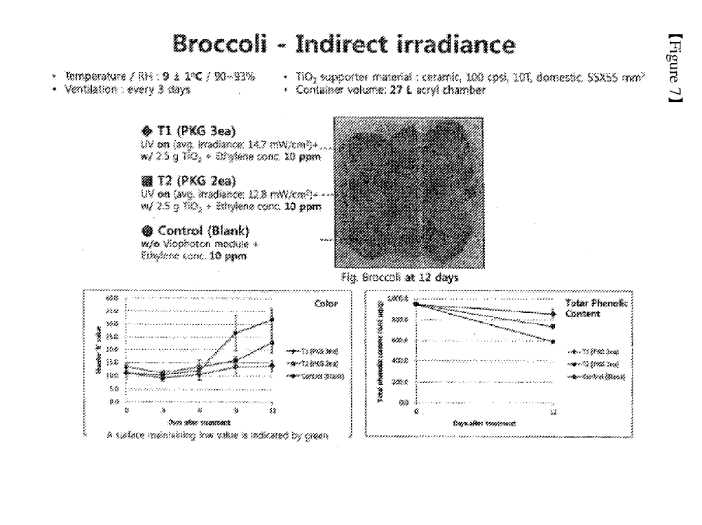

FIG. 6 is a graph depicting results of an experiment of ethylene removal performance of a multifunctional photocatalytic module according to an embodiment of the disclosed invention.

Ethylene is a gas that causes fresh food such as vegetables to be softened too earlier. In this experiment, a time taken to remove ethylene was measured in a closed space of 27 L at 5° C. The configurations of the photocatalytic filter and the ultraviolet light source are the same as in the experiment of FIG. 5. The results of the experiments differed from each other to some degree, but exhibited excellent ethylene removal performances. On the other hand, the ionizer rarely had a removal effect. That is, it has been found that the multifunctional photocatalytic module of the disclosed invention has high freshness maintenance efficiency for fresh food.

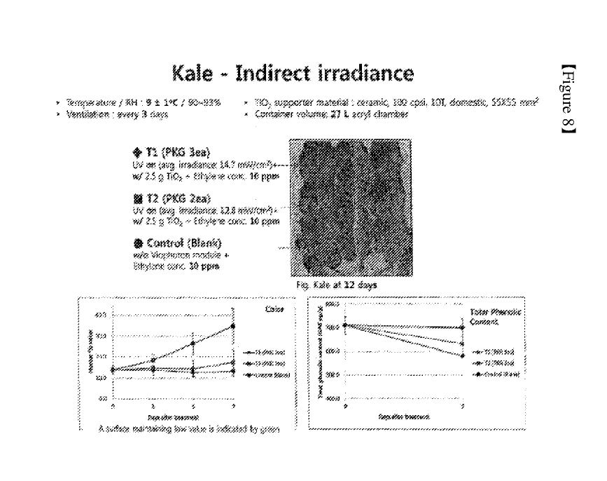

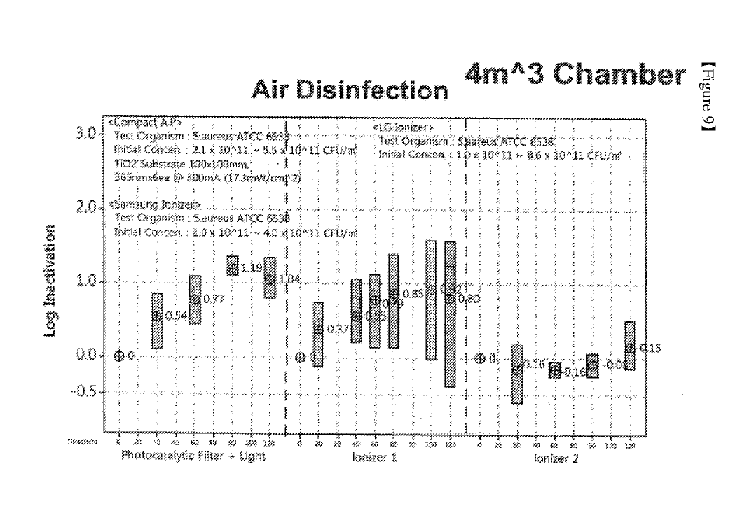

FIG. 7 is a graph depicting results of an experiment of freshness maintenance performance of a multifunctional photocatalytic module conducted for broccoli according to an embodiment of the disclosed invention. FIG. 8 is a graph depicting results of an experiment of freshness maintenance performance of a multifunctional photocatalytic module conducted for kale according to an embodiment of the disclosed invention.

The graphs depict results obtained by measuring change in condition of broccoli and kale in a space of 27 L at a temperature between about 8° C and about 10° C with different intensities of ultraviolet light radiated onto the photocatalytic filter formed by applying TiO₂ onto a ceramic base which has a thickness of 10 mm and a surface area of 55² mm² and contains about 100 cells per square inches and without a photocatalytic filter and a ultraviolet light source provided. It can be seen from the results of the experiments that using the multifunctional photocatalytic module rarely causes yellowing and maintains almost the same total phenolic content. Similar to the experimental results of FIG. 6, these results support that the multifunctional photocatalytic module of the disclosed invention has high freshness maintenance efficiency for fresh food.

FIG. 9 is a graph depicting results of an experiment of air sterilization performance of a multifunctional photocatalytic module according to an embodiment of the disclosed invention. As can be seen from the results of FIG. 9, when 6 UV LEDs are used to radiate ultraviolet light having a peak wavelength of 365 nm and intensity of about 17.3 mW/cm² on the surface of the photocatalytic filter coated with TiO₂, sterilization effect is more excellent than when the ionizer is used. The results suggest that the technique of destroying cell membranes of germs or viruses through photocatalytic reaction is very valid in an environment such as the refrigerator.

Thus far, exemplary embodiments of the disclosed invention have been described in detail with reference to the accompanying drawings. However, the disclosed invention is not limited to the exemplary embodiments, and it is apparent that modifications and variations can be made within the scope of the disclosed invention. Effects of the disclosed invention which are not explicitly described above but are predictable from the configuration of the disclosed invention will be apparent to those skilled in the art from the above descriptions.

1. A multifunctional photocatalytic module comprising:
a duct providing an air passage through which air flows and having a first side, a second side opposing to the first side, and a third side connecting the first side and the second side, the first side and the second side having a size greater than the third side;
a suction port and a discharge that are formed on opposite sides of the duct;
a fan disposed in the duct to be closer to the suction port than the discharge port, the fan introducing air from the suction port and applying pressure to air to move toward the discharge port;
a photocatalytic filter disposed in the duct to be closer to the discharge port than the suction port; and
a light source disposed between the photocatalytic filter and the fan and configured to radiate ultraviolet light toward the photocatalytic filter,
wherein the fan is inclined at a predetermined angle with respect to a longitudinal direction of the duct and structured to discharge air in a direction inclined with respect to the longitudinal direction of the duct.

2. The multifunctional photocatalytic module according to claim 1, wherein the inclination angle is within a range between 30° and 60°.

3. The multifunctional photocatalytic module according to claim 1, wherein the fan is structured to discharge air by applying pressure to the air in a direction directed to a surface of the first side of the duct.

4. The multifunctional photocatalytic module according to claim 3, wherein the fan has a flat shape and inclined in a direction along the first or the second side.

5. The multifunctional photocatalytic module according to claim 1, wherein the light source is installed between the fan and the discharge port, and comprises at least one ultraviolet (UV) light emitting diode (LED) provided on a substrate.

6. The multifunctional photocatalytic module according to claim 5, wherein a peak wavelength of the UV LED is between 360 nm and 370 nm.

7. The multifunctional photocatalytic module according to claim 5, wherein the substrate is arranged across the duct such that a distance d1 between the first side and an end of the substrate is shorter than a distance d2 between the second side and the other end of the substrate.

8. The multifunctional photocatalytic module according to claim 5, wherein the substrate is spaced apart from the photocatalytic filter by a distance between 25 mm and 40 mm.

9. The multifunctional photocatalytic module according to claim 6, wherein an intensity of ultraviolet light radiated onto a surface of the photocatalytic filter facing the light source is between 12 mW/cm² and 18 mW/cm².

10. The multifunctional photocatalytic module according to claim 3, further comprising a flow guide between the photocatalytic filter and the light source, the flow guide having a shape protruding inward from the third side and providing narrower air passage than the air passage without the flow guide.

11. (canceled)

12. The multifunctional photocatalytic module according to claim 1, wherein the photocatalytic filter includes a photocatalytic material formed onto a supporter having a plurality of cells neighboring one another and providing an air flow path, an inlet of the air flow path being disposed to face the light source.

13. The multifunctional photocatalytic module according to claim 3, wherein the suction port is formed adjacent to the first or the second side.

14. The multifunctional photocatalytic module according to claim 1, further comprising a nozzle formed around the discharge port the nozzle making the air passage narrower toward the discharge port.

15. A refrigerator provided with a multifunctional photocatalytic module comprising:
a duct providing an air passage through which air flows and having a first side, a second side opposing to the first side, and a third side connecting the first side and the second side, the first side and the second side having a size greater than the third side;
a suction port and a discharge port that are formed on opposite sides of the duct;
a fan disposed in the duct to be closer to the suction port than the discharge port, the fan introducing air from the suction port and apply pressure to air to move toward the discharge port
a photocatalytic filter disposed in the duct to be closer to the discharge port than the suction port; and
a light source disposed between the photocatalytic filter and the fan and configured to radiate ultraviolet light toward the photocatalytic filter, and
wherein the fan is inclined at a predetermined angle with respect to a longitudinal direction of the duct and structured to discharge air in a direction inclined with respect to the longitudinal direction of the duct, and
wherein the suction port and the discharge port are installed to communicate with a cooling space of the refrigerator.

16. The multifunctional photocatalytic module according to claim 10, wherein the duct provides the air passage having a cross section with a square shape after air passes through the flow guide.

17. The refrigerator according to claim 15, further comprising a flow guide between the photocatalytic filter and the light source, the flow guide having a shape protruding inward from the third side and providing narrower air passage than the air passage without the flow guide

18. The refrigerator according to claim 15, wherein the inclination angle is within a range between 30° and 60°.

19. The refrigerator according to claim 15, wherein the light source provides a peak wavelength between 360 nm and 370 nm.
